(19) United States
(12) Patent Application Publication
(10) Pub. No.: US 2007/0197578 A1
(43) Pub. Date: Aug. 23, 2007

(54) Control agent containing n-substituted indole derivative for acarian parasitic on animal

(76) Inventors: Satoshi Tanabe, Kasukabe (JP); Hiroki Hotta, Saitama (JP); Tetsuya Toya, Gifu (JP); Katsuhiko Hosoda, Saitama (JP)

Correspondence Address: NIELDS & LEMACK, 176 EAST MAIN STREET, SUITE 7, WESTBORO, MA, 01581, US

(21) Appl. No.: 10/593,920

(22) PCT Filed: Apr. 22, 2004
(86) PCT No.: PCT/JP04/05770

§ 371 Date: Sep. 22, 2006

(51) Int. Cl. A61K 31/44

(52) U.S. Cl. 514/291

(57) ABSTRACT

Conventional control agents against acarians parasitic on animals do not have sufficient selective toxicity and are hence not safe for the animals to which the control agents are applied. The control agents are not always satisfactory also in control effect and quick-acting properties. Intensive studies were made on the insecticidal activity of N-substituted indole compounds against acarians and on the safety thereof for mammals including pets. As a result, it was found that an N-substituted indole derivative, e.g., 1-(2,6-dichloro-4-trifluoromethylphenyl)-3-(trifuloromethylthio)indole, or 1-(2,6-dichloro-4-trifluoromethylphenyl)-3-(dichlo-rofluoromethylthio)indole, has high insecticidal activity and quick-acting properties and is lowly toxic to mammals including pets.

### TECHNICAL FIELD

The present invention relates to an agent for controlling acarians parasitic on animals which contains an N-substituted indole derivative. This control agent is utilizable for exterminating, in particular, acarians parasitic on companion animals such as dog and cat and livestock such as cattle and pig.

### BACKGROUND ART

In recent years, the appearance rate of sanitary insect pests such as fly has been greatly reduced by the marked improvement of public hygiene, but acarians parasitic on plants and animals such as human beings, companion animals (e.g. dog and cat) and livestock (e.g. cattle and pig) are still in question. As chemicals for controlling the acarians, there are used, for example, organophosphorus insecticides, carbamate insecticides, pyrethroid insecticides, chemicals called IGR, and phenylpyrazole insecticides such as Fipronil (5-amino-1-(2,6-dichloro-4-(trifluoromethyl)phenyl)-4-((trifluoromethyl)sulfinyl)-1H-pyrazole-3-carbonitrile).

On the other hand, U.S. Pat. No. 3,290,332 and JP-A-55-151505 describe the employment of N-substituted indole derivatives as antibacterial agents.

JP-A-6-92935 describes the employment of N-substituted indole derivatives as insecticides for diamond back moth, planthoppers and the like.

In addition, JP-A-2000-26409 describes N-substituted heterocyclic substances having an aryl or heteroaryl group as the substituent, but the substituent at the 3-position of an indole ring is only a cyclic substituent in this reference.

Furthermore, U.S. Pat. No. 5,599,774 describes the employment of N-substituted indole derivatives as herbicides.

Conventional agents for controlling acarians parasitic on animals are not satisfactory in selective toxicity and are hence not safe for the animals to which the control agents are administered. The control agents are not always satisfactory also in control effect and quick-acting properties. For example, Fipronil is classified as a deleterious substance and is not sufficiently safe for the animals to which Fipronil is administered.

Under such circumstances, the present inventors earnestly investigated the insecticidal activity of N-substituted indole compounds against acarians and the safety thereof for mammals, and consequently found that a compound represented by general formula (I) has high insecticidal activity and quick-acting properties against acarians parasitic on animals and moreover, has only low toxicity to mammals, whereby the present invention has been accomplished.

### DISCLOSURE OF THE INVENTION

That is, the present invention relates to the following.
(1) An agent for controlling acarians parasitic on mammals characterized by containing an N-substituted indole derivative represented by general formula (I): 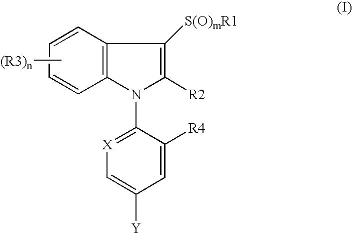
wherein X is CH, N or C-halogen atom; Y is a hydrogen atom, a C1-C5 alkyl group optionally substituted by a halogen atom(s), a C2-C5 alkenyl group optionally substituted by a halogen atom(s), a C2-C5 alkynyl group optionally substituted by a halogen atom(s), a C1-C5 alkoxyl group optionally substituted by a halogen atom(s), a halogen atom, a cyano group or a nitro group; R1 is a C1-C5 alkyl group optionally substituted by a halogen atom(s), or a C1-C5 alkoxyl group optionally substituted by a halogen atom(s); R2, R3 and R4 are independently a hydrogen atom, a C1-C5 alkyl group optionally substituted by a halogen atom(s), a C2-C5 alkenyl group optionally substituted by a halogen atom(s), a C2-C5 alkynyl group optionally substituted by a halogen atom(s), a halogen atom, a cyano group, a carboxyl group, a C1-C5 alkoxycarbonyl group optionally substituted by a halogen atom(s), a C1-C5 acyl group optionally substituted by a halogen atom(s), a nitro group, a cyanato group, a thiocyanato group, a C1-C5 alkoxyl group optionally substituted by a halogen atom(s), or S(O)ₖR5 wherein k is 0, 1 or 2 and R5 is a C1-C5 alkyl group optionally substituted by a halogen atom(s); m is 0, 1 or 2; and n is 1, 2, 3 or 4.
(2) An agent for controlling acarians according to the above item (1), wherein in general formula (I), X is N or C-halogen atom; Y is a hydrogen atom, a C1-C5 alkyl group optionally substituted by a halogen atom(s), a C1-C5 alkoxyl group optionally substituted by a halogen atom(s), or a halogen atom; R1 is a C1-C5 alkyl group optionally substituted by a halogen atom(s); R2, R3 and R4 are independently a hydrogen atom, a C1-C5 alkyl group optionally substituted by a halogen atom(s), a halogen atom, a carboxyl group, a C1-C5 alkoxycarbonyl group optionally substituted by a halogen atom(s), a C1-C5 acyl group optionally substituted by a halogen atom(s), or a C1-C5 alkoxyl group optionally substituted by a halogen atom(s); m is 0, 1 or 2; and n is 1 or 2.
(3) An agent for controlling acarians according to the above item (1), wherein in general formula (I), X is N or C—Cl ; Y is a C1-C3 alkyl group substituted by a halogen atom(s); R1 is a C1-C3 alkyl group substituted by a halogen atom(s); R2, R3 and R4 are independently a hydrogen atom, a C1-C3 alkyl group optionally substituted by a halogen atom(s), or a halogen atom; m is 0, 1 or 2; and n is 1.
(4) An agent for controlling acarians according to the above item (1), wherein the compound of general formula (I) is 1-(2,6-dichloro-4-trifluoromethylphenyl)-3-(trifluoromethyl-thio)indole or 1-(2,6-dichloro-4-trifluoromethylphenyl)-3-(dichlorofluoromethylthio)indole.
(5) An agent for controlling acarians according to any one of the above items (1) to (4), wherein the animals are companion animals.
(6) A shampoo or rinse for controlling acarians characterized by comprising an agent for controlling acarians according to any one of the above items (1) to (5).
(7) Liquid drops for controlling acarians characterized by comprising an agent for controlling acarians according to any one of the above items (1) to (5).

### BEST MODE FOR CARRYING OUT THE INVENTION

The acarian control agent of the present invention is characterized by containing an N-substituted indole derivative of the above general formula (I) wherein X is CH, N or C-halogen atom; Y is a hydrogen atom, a C1-C5 alkyl group optionally substituted by a halogen atom(s), a C2-C5 alkenyl group optionally substituted by a halogen atom(s), a C2-C5 alkynyl group optionally substituted by a halogen atom(s), a C1-C5 alkoxyl group optionally substituted by a halogen atom(s), a halogen atom, a cyano group or a nitro group; R1 is a C1-C5 alkyl group optionally substituted by a halogen atom(s), or a C1-C5 alkoxyl group optionally substituted by a halogen atom(s); R2, R3 and R4 are independently a hydrogen atom, a C1-C5 alkyl group optionally substituted by a halogen atom(s), a C2-C5 alkenyl group optionally substituted by a halogen atom(s), a C2-C5 alkynyl group optionally substituted by a halogen atom(s), a halogen atom, a cyano group, a carboxyl group, a C1-C5 alkoxycarbonyl group optionally substituted by a halogen atom(s), a C1-C5 acyl group optionally substituted by a halogen atom(s), a nitro group, a cyanato group, a thiocyanato group, a C1-C5 alkoxyl group optionally substituted by a halogen atom(s), or S(O)ₖR5 wherein k is 0, 1 or 2 and R5 is a C1-C5 alkyl group optionally substituted by a halogen atom(s); m is 0, 1 or 2; and n is 1, 2, 3 or 4.

The term “halogen atom” used herein means a fluorine atom, a chlorine atom, a bromine atom or an iodine atom. The halogen atom is preferably a fluorine atom, a chlorine atom or a bromine atom. When any of the substituents contains a plurality of halogen atoms, these halogen atoms may be the same or different.

The substituent X in general formula (I) used in the present invention is CH, N or C-halogen atom, and is particularly preferably N or C—Cl.

The C1-C5 alkyl group for Y in general formula (I) used in the present invention includes linear or branched C1-C5 alkyl groups. Specific examples thereof are methyl group, ethyl group, propyl group, isopropyl group, butyl group, tert-butyl group, pentyl group, etc. Specific examples of the C1-C5 alkyl group substituted by a halogen atom(s) are chloromethyl group, dichloromethyl group, fluoromethyl group, difluoromethyl group, trifluoromethyl group, dichlorofluoromethyl group, chlorodifluoromethyl group, trichloromethyl group, pentafluoroethyl group, etc.

The C2-C5 alkenyl group for Y in general formula (I) used in the present invention includes, for example, vinyl group, allyl group, isopropenyl group, butenyl group and pentenyl group. The C2-C5 alkenyl group substituted by a halogen atom(s) includes, for example, fluorovinyl group, chlorovinyl group, trichlorovinyl group, 3,3,3-trifluoropropenyl group, 2-bromo-2-butenyl group and perfluoro-2-methyl-2-pentenyl group.

The C2-C5 alkynyl group for Y in general formula (I) used in the present invention includes, for example, ethynyl group and propynyl group. The C2-C5 alkynyl group substituted by a halogen atom(s) includes, for example, chloroethynyl group and chloropropynyl group.

The C1-C5 alkoxyl group for Y in general formula (I) used in the present invention includes linear or branched C1-C5 alkoxyl groups. Specific examples thereof are methoxy group, ethoxy group, propoxy group, isopropoxy group, butoxy group, tert-butoxy group, etc. Specific examples of the C1-C5 alkoxyl group substituted by a halogen atom(s) are chloro-methoxy group, bromomethoxy group, dichlorofluoromethoxy group, trifluoromethoxy group, trifluoroethoxy group, pentafluoroethoxy group, etc.

Y in general formula (I) is preferably a hydrogen atom, a C1-C5 alkyl group optionally substituted by a halogen atom(s), a C1-C5 alkoxyl group optionally substituted by a halogen atom(s), or a halogen atom, is particularly preferably a halogen atom or a C1-C3 alkyl group optionally substituted by a halogen atom(s), and is more preferably a chlorine atom, a bromine atom or a trifluoromethyl group.

The C1-C5 alkyl group optionally substituted by a halogen atom(s) for R1 in general formula (I) which is used in the present invention includes the same groups as those exemplified above as each of the C1-C5 alkyl group for Y and the C1-C5 alkyl group substituted by a halogen atom(s) for Y. Specific examples thereof are also the same as those given above in the case of Y.

The C1-C5 alkoxyl group optionally substituted by a halogen atom(s) for R1 in general formula (I) which is used in the present invention includes the same groups as those exemplified above as each of the C1 -C5 alkoxyl group for Y and the C1-C5 alkoxyl group substituted by a halogen atom(s) for Y. Specific examples thereof are also the same as those given above in the case of Y.

R1 in general formula (I) is preferably a C1-C5 alkyl group optionally substituted by a halogen atom(s), in particular, a C1-C3 alkyl group substituted by a halogen atom(s). Specific examples thereof are trifluoromethyl group, dichlorofluoromethyl group, chlorodifluoromethyl group and trichloromethyl group.

The C1-C5 alkyl group optionally substituted by a halogen atom(s) for each of R2, R3 and R4 in general formula (I) which is used in the present invention includes the same groups as those exemplified above as each of the C1-C5 alkyl group for Y and the C1-C5 alkyl group substituted by a halogen atom(s) for Y. Specific examples thereof are also the same as those given above in the case of Y.

The C2-C5 alkenyl group optionally substituted by a halogen atom(s) for each of R2, R3 and R4 in general formula (I) which is used in the present invention includes the same groups as those exemplified above as each of the C2-C5 alkenyl group for Y and the C2-C5 alkenyl group substituted by a halogen atom(s) for Y. Specific examples thereof are also the same as those given above in the case of Y.

The C2-C5 alkynyl group optionally substituted by a halogen atom(s) for each of R2, R3 and R4 in general formula (I) which is used in the present invention includes the same groups as those exemplified above as each of the C2-C5 alkynyl group for Y and the C2-C5 alkynyl group substituted by a halogen atom(s) for Y. Specific examples thereof are also the same as those given above in the case of Y.

The C1-C5 alkoxycarbonyl group optionally substituted by a halogen atom(s) for each of R2, R3 and R4 in general formula (I) which is used in the present invention includes, for example, methoxycarbonyl group, ethoxycarbonyl group, propoxycarbonyl group, butoxycarbonyl group, tert-butoxycarbonyl group and 2,2,2-trifluoroethoxycarbonyl group.

The C1-C5 acyl group optionally substituted by a halogen atom(s) for each of R2, R3 and R4 in general formula (I) which is used in the present invention includes, for example, formyl group, acetyl group, propionyl group, butyryl group, isobutyryl group, valeryl group, pivaloyl group, trifluoroacetyl group, trichloroacetyl group and 3,3,3-trifluoropropionyl group.

The C1-C5 alkoxyl group optionally substituted by a halogen atom(s) for each of R2, R3 and R4 in general formula (I) which is used in the present invention includes the same groups as those exemplified above as each of the C1-C5 alkoxyl group for Y and the C1-C5 alkoxyl group substituted by a halogen atom(s) for Y. Specific examples thereof are also the same as those given above in the case of Y.

The C1-C5 alkyl group optionally substituted by a halogen atom(s) for R5 in S(O)ₖR5 for each of R2, R3 and R4 in general formula (I) which is used in the present invention includes the same groups as those exemplified above as each of the C1-C5 alkyl group for Y and the C1-C5 alkyl group substituted by a halogen atom(s) for Y. Specific examples thereof are also the same as those given above in the case of Y. In addition, k may be 0, 1 or 2.

R2 in general formula (I) is preferably a hydrogen atom, an unsubstituted C1-C5 alkyl group or a halogen atom, and is particularly preferably a hydrogen atom or a methyl group.

R3 in general formula (I) is preferably a hydrogen atom, a C1-C5 alkoxyl group optionally substituted by a halogen atom(s), a halogen atom or a cyano group, and is particularly preferably a hydrogen atom, a fluorine atom, a chlorine atom, a bromine atom, a methoxy group or a cyano group. The substitution position of R3 is preferably the 4-, 5- or 6-position of the indole ring, in particular, the 5-position.

R4 in general formula (I) is preferably a halogen atom, a C1-C5 alkyl group optionally substituted by a halogen atom(s), or a C1-C5 alkoxyl group optionally substituted by a halogen atom(s), and is particularly preferably a chlorine atom, a fluorine atom, a trifluoromethyl group or a trifluoromethoxy group.

Although the integer m in general formula (I) used in the present invention may be 0, 1 or 2, it is preferably 0 or 2.

Although the integer n in general formula (I) used in the present invention may be 1, 2, 3 or 4, it is preferably 1 or 2, in particular, 1.

The compound of general formula (I) used in the acarian control agent of the present invention includes 1-(2,6-dichloro-4-trifluoromethylphenyl)-3-(dichlorofluoro-methylthio)indole, 1-(2,6-dichloro-4-trifluoromethylphenyl)-3-(trifluoromethylthio)indole and the like. In particular, 1-(2,6-dichloro-4-trifluoromethylphenyl)-3-(trifluoromethyl-thio)indole is preferable.

When the compound of the above general formula (I) is used as an acarian control agent, the N-substituted indole derivative may be used alone as it is, though it is preferably administered to the whole or a part of a living body to be treated, by any of, for example, the following various methods acceptable to parasiticides in order to control parasites more easily and effectively: a method of using the derivative in the form of liquid drops, a solution, a spray, a foamy preparation, tablets, granules, fine granules, a powder, capsules, an injection, a suppository, a chewable preparation or the like; a method of using the derivative in admixture with a shampoo or a rinse; a method of using the derivative by its incorporation into a collar; and a method of using the derivative in admixture with feed. Of such preparation forms, the liquid drops and the shampoo or rinse are especially preferable.

For example, the liquid drops are a liquid percutaneous preparation containing 0.1 to 20 parts by weight of the N-substituted indole derivative and 10 to 95 parts by weight of a glycol or a glycol monoalkyl ether. If necessary, other components may be properly incorporated into the liquid drops. As the other components, there are exemplified liquid carriers easily miscible with the glycol or glycol monoalkyl ether, such as alcohols (e.g. methanol, ethanol, isopropanol, tert-butanol and benzyl alcohol), propylene carbonate, N-methyl-2-pyrrolidone, water, etc.

The liquid drops are usually administered to a mammal by a topical treatment method such as spot-on treatment or pour-on treatment. The administration permits efficient control of external parasites of the mammal.

The spot-on treatment method is a method in which the external parasites are controlled by dropping a liquid agent for controlling the external parasites, for example, onto the skin at the back of the shoulder blade of the animal.

The pour-on treatment method is a method in which a liquid agent for controlling the external parasites is poured along the dorsal midline of the animal and then this control agent spreads over the surface of the body, whereby the external parasites are controlled.

The amount of the control agent administered to the animal is usually, for example, 0.001 ml/kg to 10 ml/kg in terms of a composition and is, for example, 0.1 mg/kg to 3000 mg/kg in terms of the N-substituted indole derivative.

For example, the spray is a liquid agent for controlling external parasites which contains 0.1 to 20 parts by weight of the N-substituted indole derivative and 10 to 95 parts by weight of a glycol or a glycol monoalkyl ether, an alcohol and a surfactant. If necessary, the spray may properly contain other components. The glycol or glycol monoalkyl ether includes, for example, diethylene glycol monoethyl ether and propylene glycol. The alcohol includes, for example, methanol, ethanol, isopropanol, tert-butanol and benzyl alcohol. The surfactant includes, for example, anionic surfactants, cationic surfactants and amphoteric surfactants, such as sodium higher alcohol sulfate, stearylmethyl-ammonium chloride, polyoxyethylene alkylphenyl ether, laurylbetaine, etc. The amount of this control agent administered to an animal per kg of the animal is usually about 0.01 ml/kg to about 10 ml/kg in terms of a composition and about 0.1 mg/kg to about 3000 mg/kg in terms of the N-substituted indole derivative.

The capsules, pills or tablets may be prepared by properly dividing the N-substituted indole derivative, mixing the derivative with a diluent or a carrier, adding thereto a disintegrating agent and/or a binder, such as starch, lactose, talc, magnesium stearate or the like, and if necessary, compressing the resulting mixture into tablets.

The injection should be prepared as a sterile solution. The sterile solution may contain other substances such as a salt or glucose in an amount sufficient to make the solution isotonic with regard to blood. A liquid carrier usable in the injection includes vegetable oils such as sesame oil, etc.; glycerides such as triacetin, etc.; and esters such as benzyl benzoate, isopropyl myristate, fatty acid derivatives of propylene glycol, etc., as well as organic solvents such as pyrrolidone, glycerol formal, etc. This pharmaceutical composition is prepared by dissolving or suspending the active ingredient in the above-exemplified liquid carrier so that the composition may contain the active ingredient in an amount of, for example, 0.01 to 10% by weight.

As to the method of using the N-substituted indole derivative in admixture with a shampoo or a rinse, such a composition may be prepared by incorporating the N-substituted indole derivative into a commercial shampoo or rinse in an amount of 0.01 to 10%, preferably 0.1 to 2%. In addition, it is also possible to prepare a shampoo or rinse for exclusive use comprising the components of a conventional shampoo or rinse for animals and the N-substituted indole derivative. The concentration of the N-substituted indole derivative in the shampoo or rinse for exclusive use is about 0.01 to about 10%, preferably about 0.1 to about 2%. Specifically, the shampoo or rinse for exclusive use is prepared, for example, from the N-substituted indole derivative, an acceptable solvent, a solubilizer or an emulsifier, a wash or a treatment, water and the like. The shampoo or rinse for exclusive use may further contain an aromatic, a thickening agent or a viscosity modifier, a pH adjuster and the like. The acceptable solvent includes, for example, glycols or glycol monoalkyl ethers, and alcohols such as methanol, ethanol, isopropanol, tert-butanol, benzyl alcohol, etc.

The other pharmaceutical compositions may also be prepared by adding components which are considered necessary for preparing the compositions, such as generally known surfactants, diluents, additives, stabilizers, etc.

In addition, the acarian control agent of the present invention may be administered together with animal feed. For this administration, concentrated feed containing the control agent or a premix may be prepared.

The acarian control agent of the present invention may be mixed and used together with not only other insecticides, nematicides and other acaricides but also synergists and the like. As these chemicals, there are used, for example, organophosphorus compounds such as Diazinon, DDVP (2,2-dichlorovinyl-O,O-dimethyl phosphate), etc.; carbamate compounds such as Carbosulfan, etc.; pyrethroid compounds such as Cycloprothrin, Ethofenprox, Allethrin, Permethrin, etc.; chloronicotinyl compounds such as Imidacloprid, etc.; phenylpyrazole compounds such as Fipronil, etc.; benzoylurea compounds such as Lufenuron, etc.; juvenile-hormone-like compounds such as Methoprene, Pyriproxyfen, etc.; hydrazine compounds such as Chromafenozide, Tebufenozide, etc.; macrolide compounds such as Milbemectin, Ivermectin, Moxydectin, Seramectin, etc.; Buprofezin; and Azadirachtin.

As to the administration methods of the above-mentioned pharmaceutical compositions, the compositions may be administered by conventional methods, respectively. In particular, the amount of the composition administered to an animal is not particularly limited so long as it is effective in controlling acarians without side effects. It is usually about 0.01 mg/kg to about 3000 mg/kg, preferably about 0.1 mg/kg to about 1500 mg/kg, particularly preferably about 1 mg/kg to about 500 mg/kg.

The interval between administrations of the acarian control agent of the present invention may be set on the basis of a period during which the active ingredient of the control agent remains in an effective amount on or in a living thing to which the control agent is administered, and it can exhibit the desired effect sufficiently. The interval is varied depending on the kind of the living thing, the compound used and the pharmaceutical form. For example, in the case of the liquid drops, the interval between administrations is about 1 month to about 1 year, preferably about 1 month to about 6 months, particularly preferably about 1 month to about 3 months.

Acarians controllable by the acarian control agent of the present invention are not particularly limited so long as they are classified as Acarina belonging to Arachnida among Arthropoda and are parasitic on mammals. The control agent is effective against, in particular, acarians parasitic on companion animals such as dog and cat and livestock such as cattle and pig. Specific examples of the controllable acarians are Haemaphysalis including Haemaphysalis longicornis, Haemaphysalis flava, Reachichimadani, etc.; Ixodidae including Boophilus microplus, Ixodes ovatus, Ixodes ricinus, New South Wales tick, Ixodes scaprilas, etc.; Amblyomma including Amblyomma hebraeum, Amblyomma amerocanum, etc.; Rhipicephalus including Rhipicephalus sanguineus, Rhipicephalus cimus, etc.; Dermacentor including Dermacentor variabilis, etc.; Otodectes spp.; Ornithonyssus spp.; Trombicula spp.; Sarcoptes spp.; Notoedres spp.; and the like. In particular, Haemaphysalis longicornis, Boophilus microplus and the like are exemplified as the controllable acarians.

The companion animals refer to dogs, cats, hamsters, rabbits and the like, which are commonly kept by households.

Next, typical examples of the compound of the above general formula (I) used in the present invention are listed in Table 1.

TABLE 1

| No. | X | Y | m | R1 | R2 | R3 | M | n |
|---|---|---|---|---|---|---|---|---|
| 1 | N | CF3 | 0 | CCl2F | H | H | Cl | 1 |
| 2 | N | CF3 | 0 | CCl2F | H | 5-F | Cl | 1 |
| 3 | N | CF3 | 0 | CCl2F | H | 5-Cl | Cl | 1 |
| 4 | N | CF3 | 0 | CCl2F | H | 5-Br | Cl | 1 |
| 5 | N | CF3 | 0 | CCl2F | H | 5-OCH3 | Cl | 1 |
| 6 | N | CF3 | 0 | CCl2F | H | 5-CN | Cl | 1 |
| 7 | N | CF3 | 0 | CCl2F | H | 4-Cl | Cl | 1 |
| 8 | N | CF3 | 0 | CCl2F | H | 6-Cl | Cl | 1 |
| 9 | N | CF3 | 0 | CF3 | H | H | Cl | |
| 10 | N | CF3 | 0 | CF3 | H | 5-Cl | Cl | 1 |
| 11 | N | CF3 | 0 | CCl3 | H | H | Cl | 1 |
| 12 | N | CF3 | 0 | CCl3 | H | 5-Cl | Cl | 1 |
| 13 | N | Cl | 0 | CCl2F | H | H | Cl | 1 |
| 14 | N | CF3 | 0 | CCl2F | CH3 | H | Cl | 1 |
| 15 | N | CF3 | 1 | CCl2F | H | H | Cl | 1 |
| 16 | N | CF3 | 2 | CCl2F | H | H | Cl | 1 |
| 17 | CCl | CF3 | 0 | CCl2F | H | H | Cl | 1 |
| 18 | CCl | CF3 | 0 | CCl2F | H | 5-F | Cl | 1 |
| 19 | CCl | CF3 | 0 | CCl2F | H | 5-Cl | Cl | 1 |
| 20 | CCl | CF3 | 0 | CCl2F | H | 5-Br | Cl | 1 |
| 21 | CCl | CF3 | 0 | CCl2F | H | 5-OCH3 | Cl | 1 |
| 22 | CCl | CF3 | 0 | CCl2F | H | 5-CN | Cl | 1 |
| 23 | CCl | CF3 | 0 | CCl2F | H | 4-Cl | Cl | 1 |
| 24 | CCl | CF3 | 0 | CCl2F | H | 6-Cl | Cl | 1 |
| 25 | CCl | CF3 | 0 | CF3 | H | H | Cl | 1 |
| 26 | CCl | CF3 | 0 | CF3 | H | 5-Cl | Cl | 1 |
| 27 | CCl | CF3 | 0 | CCl3 | H | H | Cl | 1 |
| 28 | CCl | CF3 | 0 | CCl3 | H | 5-Cl | Cl | 1 |
| 29 | CCl | Cl | 0 | CCl2F | H | H | Cl | 1 |
| 30 | CCl | CF3 | 0 | CCl2F | CH3 | H | Cl | 1 |
| 31 | CCl | CF3 | 1 | CCl2F | H | H | Cl | 1 |
| 32 | CCl | CF3 | 2 | CCl2F | H | H | Cl | 1 |

### TEST EXAMPLES

Acarian control tests using the acarian control agent of the present invention and a toxicity test on mice are described below.

### Test Example 1

### In vitro Control Effect on Acarians obtained by the use of an N-substituted indole derivative

Neoglamine was added to the emulsion of Example 1 in an amount of 0.01% and the resulting mixture was diluted with tap water to each concentration shown in Table 2. After a commercial 0.5×15 cm Pasteur's pipette was immersed in the dilution for 30 seconds, it was vertically stood on cotton and air-dried. The head of the air-dried Pasteur's pipette was plugged with cotton, and 10 hatched larvae of Haemaphysalis longicornis were sucked from the cotton-plugged end side by the use of a suction pump and then the other end was sealed with putty. After the suction, the pipette was allowed to stand in a desiccator containing a saturated aqueous disodium hydrogenphosphate solution and maintained at 23° C. Observation was carried out after 2 days and after 4 days. Fipronil was used as a positive control.

TABLE 2

| | | Mortality | Mortality |
|---|---|---|---|
| Compound | Dose (ppm) | after 2 days | after 4 days |
| 17 | 10 | 100 | 100 |
| | 1 | 20 | 80 |
| | 0.1 | 0 | 0 |
| 25 | 10 | 100 | 100 |
| | 1 | 100 | 100 |
| | 0.1 | 100 | 100 |
| | 0.01 | 30 | 60 |
| Fipronil | 10 | 100 | 100 |
| | 1 | 100 | 100 |
| | 0.1 | 30 | 60 |

As can be seen from the results shown in Table 2, the N-substituted indole derivative as compound No. 25 showed a mortality of Haemaphysalis longicornis of 100% after 2 days at a concentration of as low as 0.1 ppm. This fact indicates the high insecticidal activity and quick-acting properties of the N-substituted indole derivative.

### Test Example 2

### In vivo Control Effect Obtained by the Use of an N-Substituted Derivative (No. 17) in the Case Rabbit

About 40 hatched larvae of Haemaphysalis longicornis were inoculated on the ears of a rabbit by the use of a cloth bag, made parasitic on the rabbit and allowed to suck the blood. After 24 hours, the cloth bag was taken off and ticks sucking the blood were counted. The right ear was subjected to spot-on treatment with 0.1 ml of a solution prepared by dissolving compound No. 17 in a base ingredient for preparing liquid drops (a mixed solution consisting of 75 parts by weight of diethylene glycol monoethyl ether and 15 parts by weight of ethanol) to a concentration of 20%. The left ear was not treated. Three hours, 1 day and 2 days after the spot-on treatment, the living ticks on each of the right and left ears were counted. As a control animal, there was used a rabbit whose right ear had been treated with only the base ingredient for preparation for spot-on treatment and whose left ear had been not treated. Table 3 shows the results obtained.

TABLE 3

| | | Number of living ticks | | | |
|---|---|---|---|---|---|
| | | Before | After | After | After |
| Compound | Ear | dropping | 3 hours | 1 day | 2 days |
| 17 | Right ear | 15 | 3 | 1 | 0 |
| | Left ear | 15 | 15 | 0 | 0 |
| Control | Right ear | 20 | 20 | 17 | 17 |
| | Left ear | 56 | 56 | 33 | 33 |

As can be seen from the results shown in Table 3, compound No. 17 quickly exterminated ticks on the right ear in 3 hours after the treatment. Furthermore, in 1 day after the treatment, this compound also exterminated ticks on the left ear, the untreated ear to annihilate the ticks on the ears. These facts indicate that compound No. 17 quickly exterminates ticks and spreads rapidly on the body of the animal.

### Test Example 3

### Toxicity of N-Substituted Indole Derivatives to Mouse

The compound listed in Table 1 or Fipronil was dissolved in olive oil to a predetermined concentration, and the resulting solution was directly administered into the stomachs of std:ddy strain male mice by the use of a probe. The dose was 30 mg/kg or 100 mg/kg. Whether the mice were alive or dead was observed 3 hours, 1 day, 7 days and 14 days after the administration. Table 4 shows the test results obtained for compounds Nos. 14, 17 and 25 listed in Table 1.

TABLE 4

| | | Cumulative mortality | | | |
|---|---|---|---|---|---|
| | | (number of deaths/number of test animals) | | | |
| | Dose | After | After | After | After |
| Compound No. | (mg/kg) | 3 hours | 1 day | 7 days | 14 days |
| 14 | 30 | 0/5 | 0/5 | 0/5 | 0/5 |
| | 100 | 0/5 | 0/5 | 0/5 | 0/5 |
| 17 | 30 | 0/5 | 0/5 | 0/5 | 0/5 |
| | 100 | 0/5 | 0/5 | 0/5 | 0/5 |
| 25 | 30 | 0/5 | 0/5 | 0/5 | 0/5 |
| | 100 | 0/5 | 0/5 | 0/5 | 0/5 |
| Fipronil | 30 | 0/5 | 1/5 | 1/5 | 1/5 |
| | 100 | 1/5 | 5/5 | 5/5 | 5/5 |

As can be seen from the results shown in Table 4, this test indicates that the N-substituted indole derivatives have only low toxicity to mouse.

### Test Example 4

### Administration of an N-Substituted Indole Derivative (No. 17) to a Cat

Compound No. 17 was dissolved in a base ingredient for preparation for spot-on treatment (a mixed solution consisting of 75 parts by weight of diethylene glycol monoethyl ether and 15 parts by weight of ethanol) to a concentration of 10%, 20% or 30%, and 0.5 ml of the resulting solution was dropped on the back of the shoulder blade of a cat. After the dropping, the clinical symptom of the cat was observed. Table 5 shows the test results.

TABLE 5

| Compound | Dropping concentration (%) | Clinical symptom |
|---|---|---|
| 17 | 10 | No sign was recognized |
| | 20 | No sign was recognized |
| | 30 | No sign was recognized |

As can be seen from the results shown in Table 5, no abnormal sign due to the spot-on dropping of the solution for 10, 20 or 30% liquid drops of compound No. 17 was recognized, namely, no influence of the administration of the agent was recognized. This fact indicates that compound No. 17 has only low toxicity to cat.

### Test Example 5

### (Comparative Test) Insecticidal Effect of N-Substituted Indole Derivatives on Two-Spotted Spider Mite (Tetranychus urticae)

Twenty adult female two-spotted spider mites were released on the leaves of a potted kidney bean plant. One day after the release, the kidney bean leaves were immersed for several seconds in a chemical solution obtained by diluting a 20% emulsion of each compound with tap water to a concentration of 200 ppm. After air-dryness, the leaves were placed in a room thermostated at 25° C. Two days after the immersion, whether the adult two-spotted spider mites on the leaves were alive or dead were judged and the dead and alive were counted. The mortality was calculated. Table 6 shows the results obtained.

TABLE 6

| | | Mortality |
|---|---|---|
| Compound | Treating concentration (ppm) | after 2 days (%) |
| No. 17 | 200 | 0 |
| No. 25 | 200 | 0 |

As can be seen from the results shown in Table 6, compounds No. 17 and No. 25 had no insecticidal activity against the two-spotted spider mites.

### EXAMPLES

Formulation examples are described below as working examples but these working examples are not intended in any way to limit the scope of the present invention.

### Example 1

### Emulsion

Eighty-five parts by weight of dimethyl sulfoxide, 85 parts by weight of xylene and 20 parts by weight of Newcalgen 900 (mfd. by Takemoto Oil Fat Co., Ltd.) were mixed to effect dissolution. Ninety parts by weight of the resulting mixed solution was mixed with 10 parts by weight of compound No. 17 or No. 25 listed in Table 1, to obtain an emulsion.

### Example 2

### Liquid Drops

Seventy-five parts by weight of diethylene glycol monoethyl ether and 15 parts by weight of ethanol were mixed to effect dissolution. Eighty parts by weight of the resulting mixed solution was mixed with 20 parts by weight of compound No. 17 or No. 25 to obtain 20% liquid drops for spot-on treatment. In the same manner as above, 10% and 30% liquid drops for spot-on treatment were also prepared.

### Example 3

### Shampoo.Rinse

Compound No. 25 listed in Table 1 was added to a commercial shampoo or rinse for dog or cat in an amount of 1% and sufficiently stirred to obtain a homogeneous mixture. Thus, a shampoo for controlling acarians or a rinse for controlling acarians was obtained.

### INDUSTRIAL APPLICABILITY

The acarian control agent containing an N-substituted indole derivative of the present invention has a low insecticidal activity against acarians parasitic on plants, but it has control effect and quick-acting properties with respect to acarians parasitic on animals and is very good at controlling, in particular, acarians parasitic on companion animals such as dog, rabbit, cat, etc. and livestock. The quick-acting properties of the control agent mean that animals treated with the control agent are hardly infected with diseases carried by acarians, and the like. Furthermore, the acarian control agent of the present invention is very useful because it has only low toxicity to mammals including pets.

1. An agent for controlling acarians parasitic on mammals characterized by containing an N-substituted indole derivative represented by general formula (I): 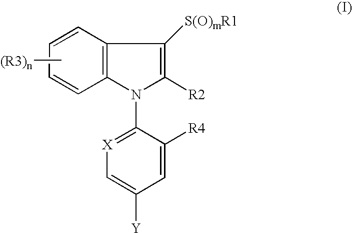
wherein X is CH, N or C-halogen atom; Y is a hydrogen atom, a C1-C5 alkyl group optionally substituted by a halogen atom(s), a C2-C5 alkenyl group optionally substituted by a halogen atom(s), a C2-C5 alkynyl group optionally substituted by a halogen atom(s), a C1-C5 alkoxyl group optionally substituted by a halogen atom(s), a halogen atom, a cyano group or a nitro group; R1 is a C1-C5 alkyl group optionally substituted by a halogen atom(s), or a C1-C5 alkoxyl group optionally substituted by a halogen atom(s); R2, R3 and R4 are independently a hydrogen atom, a C1-C5 alkyl group optionally substituted by a halogen atom(s), a C2-C5 alkenyl group optionally substituted by a halogen atom(s), a C2-C5 alkynyl group optionally substituted by a halogen atom(s), a halogen atom, a cyano group, a carboxyl group, a C1-C5 alkoxycarbonyl group optionally substituted by a halogen atom(s), a C1-C5 acyl group optionally substituted by a halogen atom(s), a nitro group, a cyanato group, a thiocyanato group, a C1-C5 alkoxyl group optionally substituted by a halogen atom(s), or S(O)ₖR5 wherein k is 0, 1 or 2 and R5 is a C1-C5 alkyl group optionally substituted by a halogen atom(s); m is 0, 1 or 2; and n is 1, 2, 3 or 4.

2. An agent for controlling acarians according to claim 1, wherein in general formula (I), X is N or C-halogen atom; Y is a hydrogen atom, a C1-C5 alkyl group optionally substituted by a halogen atom(s), a C1-C5 alkoxyl group optionally substituted by a halogen atom(s), or a halogen atom; R1 is a C1-C5 alkyl group optionally substituted by a halogen atom(s); R2, R3 and R4 are independently a hydrogen atom, a C1-C5 alkyl group optionally substituted by a halogen atom(s), a halogen atom, a carboxyl group, a C1-C5 alkoxycarbonyl group optionally substituted by a halogen atom(s), a C1-C5 acyl group optionally substituted by a halogen atom(s), or a C1-C5 alkoxyl group optionally substituted by a halogen atom(s); m is 0, 1 or 2; and n is 1 or 2.

3. An agent for controlling acarians according to claim 1, wherein in general formula (I), X is N or C—Cl; Y is a C1-C3 alkyl group substituted by a halogen atom(s); R1 is a C1-C3 alkyl group substituted by a halogen atom(s); R2, R3 and R4 are independently a hydrogen atom, a C1-C3 alkyl group optionally substituted by a halogen atom(s), or a halogen atom; m is 0, 1 or 2; and n is 1.

4. An agent for controlling acarians according to claim 1, wherein the compound of general formula (I) is 1-(2,6-dichloro-4-trifluoromethylphenyl)-3-(trifluoromethyl-thio)indole or 1-(2,6-dichloro-4-trifluoromethylphenyl)-3-(dichlorofluoromethylthio)indole.

5. An agent for controlling acarians according to any one of claims 1 to 4, wherein the animals are companion animals.

6. A shampoo or rinse for controlling acarians characterized by comprising an agent for controlling acarians according to any one of claims 1 to 5.

7. Liquid drops for controlling acarians characterized by comprising an agent for controlling acarians according to any one of claims 1 to 5.
